(19) United States
(12) Patent Application Publication
(10) Pub. No.: US 2024/0021282 A1
(43) Pub. Date: Jan. 18, 2024

(54) MEDICAL TREATMENT SYSTEM

(71) Applicant: KONINKLIJKE PHILIPS N.V., EINDHOVEN (NL)

(72) Inventors: CORNELIS CONRADUS ADRIANUS MARIA VAN ZON, EINDHOVEN (NL); IKARO GARCIA ARAUJO DA SILVA, OOSTERHOUT (NL); MAHMOUDREZA SHARIFI, UTRECHT (NL); KYLE JOSEPH TROUT, CAMBRIDGE, MA (US)

(21) Appl. No.: 18/033,389

(22) PCT Filed: Oct. 22, 2021
(86) PCT No.: PCT/EP2021/079392

§ 371 Date: Apr. 24, 2023

Related U.S. Application Data
(60) Provisional application No. 63/104,641, filed on Oct. 23, 2020.

(51) Int. Cl. G16H 20/00; G16H 40/63

(52) U.S. Cl. CPC G16H 20/00; G16H 40/63

(57) ABSTRACT

A system for treating a patient in a medical procedure includes a computer and a medical apparatus. The computer includes a memory that stores instructions and a processor that executes the instructions. The medical apparatus is configured to apply a medical treatment to the patient when instructed to do so by the computer. The instructions cause the system to obtain medical data of the patient indicative of a medical condition to be treated, and select an algorithm and apply the algorithm to the medical data to identify the medical treatment to remedy the medical condition. The instructions also cause the system to determine whether it is authorized to apply the medical treatment to the patient and, if so, instruct the medical apparatus to apply the medical treatment to the patient. The medical apparatus applies the medical treatment to the patient when instructed to do so.

### BACKGROUND

Clinical Decision Support (CDS) refers to computer-based support for clinical staff responsible for making decisions for the care of patients. Computer-based support for clinical decision-making staff is widespread and can take many forms, from patient-specific visual/numeric health status indicators to patient-specific health status predictions and patient-specific health care recommendations. CDS has steadily been accepted into mainstream healthcare, and this may be due in part to CDS only providing decision-making support and not being used as a substitute for clinical staff decision-making.

### SUMMARY

According to an aspect of the present disclosure, a system for treating a patient in a medical procedure includes a computer and a medical apparatus. The computer includes a memory that stores instructions and a processor that executes the instructions. The medical apparatus is configured to apply a medical treatment to the patient when instructed to apply the medical treatment by the computer. When executed by the processor, the instructions cause the system to perform a process that includes obtaining medical data of the patient indicative of a medical condition to be treated, and selecting an algorithm and applying the algorithm to the medical data to identify the medical treatment to remedy the medical condition. The process executed by the system also includes determining whether the system can be authorized to apply the medical treatment to the patient and, when the system can be authorized to apply the medical treatment to the patient, instructing the medical apparatus to apply the medical treatment to the patient. The medical apparatus applies the medical treatment to the patient based on the computer instructing the medical apparatus to apply the medical treatment to the patient.

According to another aspect of the present disclosure, a method for treating a patient in a medical procedure includes obtaining, via a computer system that includes a memory that stores instructions and a processor that executes the instructions, medical data of the patient indicative of a medical condition to be treated. The method also includes selecting, by the computer system, an algorithm and applying the algorithm to the medical data to identify a medical treatment to remedy the medical condition, and determining, by the computer system, whether the computer system can be authorized to instruct the medical apparatus to apply the medical treatment to the patient. When the computer system can be authorized to instruct the medical apparatus to apply the medical treatment to the patient, the method includes instructing the medical apparatus to apply the medical treatment to the patient. The medical apparatus applies the medical treatment to the patient based on the computer instructing the medical apparatus to apply the medical treatment to the patient.

According to yet another aspect of the present disclosure, a tangible non-transitory computer readable storage medium stores a computer program. When executed by a processor, the computer program causes a system that includes the tangible non-transitory computer readable storage medium to obtain medical data of the patient indicative of a medical condition to be treated, and to select an algorithm and apply the algorithm to the medical data to identify a medical treatment to remedy the medical condition. The computer program also causes the system to determine whether the system can be authorized to instruct the medical apparatus to apply the medical treatment to the patient. When the system can be authorized to instruct the medical apparatus to apply the medical treatment to the patient, the computer program causes the system to instruct the medical apparatus to apply the medical treatment to the patient. The medical apparatus applies the medical treatment to the patient based on the computer instructing the medical apparatus to apply the medical treatment to the patient.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a medical treatment system, in accordance with a representative embodiment.

FIG. 2 illustrates a method performed by a medical treatment system, in accordance with a representative embodiment.

FIG. 3 illustrates a hybrid of a medical treatment system and a method performed by the medical treatment system, in accordance with a representative embodiment.

FIG. 4 illustrates another method performed by a medical treatment system, in accordance with a representative embodiment.

FIG. 5 illustrates another hybrid of a medical treatment system and a method performed by the medical treatment system, in accordance with a representative embodiment, in accordance with a representative embodiment.

FIG. 6 illustrates another method performed by a medical treatment system, in accordance with a representative embodiment.

FIG. 7 illustrates another method performed by a medical treatment system, in accordance with a representative embodiment.

FIG. 8 illustrates another method performed by a medical treatment system, in accordance with a representative embodiment.

FIG. 9 illustrates another method performed by a medical treatment system, in accordance with a representative embodiment.

FIG. 10 illustrates a data set used as an input for a medical treatment system, in accordance with a representative embodiment.

FIG. 11 illustrates a computer system in a medical treatment system, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, clinical decisions can be made and implemented by a medical treatment system. The medical treatment system may also perform pre-diagnostic tasks such as placing orders for tests, diagnostic tasks such as ordering an MRI scan, and post-diagnostic tasks such as placing orders for medications, or calling a code. Notably, a pre-diagnostic task and/or a post-diagnostic task may include filing an order to move a patient to a different room, unit or hospital. The medical treatment system may be an informatics system that can optionally control associated therapeutic devices. Automated clinical decision making (ACDM) may be used to i) decide if an action must be taken or not; ii) in case an action must be taken, specify which action must be taken, iii) selectively implement the action to be taken; and, iv) document the decision, the reasoning behind the decision, and the action that was selectively implemented. Medical treatments as described herein may include bedside clinician interventions and application of various therapeutics from medical apparatuses such as oxygen supply systems, ventilators, and pharmaceutical supply mechanisms.

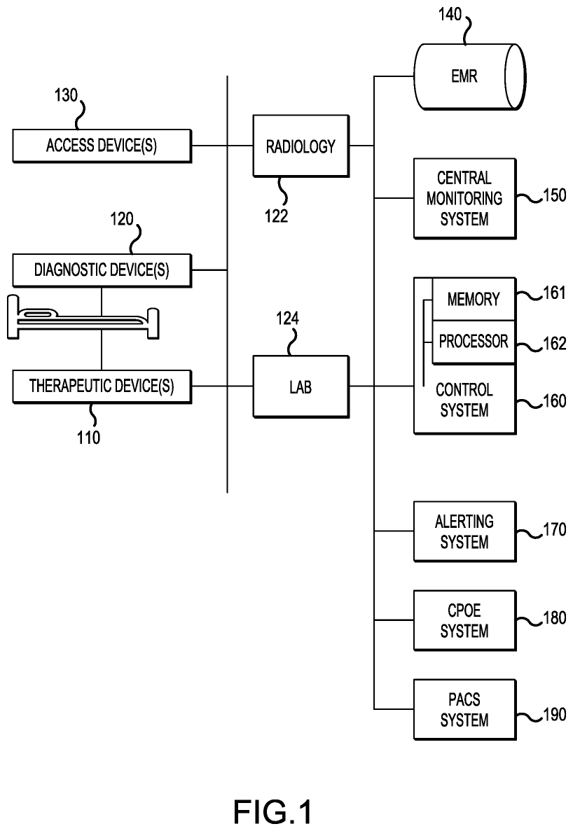

FIG. 1 illustrates a medical treatment system, in accordance with a representative embodiment.

The medical treatment system of FIG. 1 includes one or more diagnostic device(s) 120 and one or more therapeutic devices(s) 110 proximate to a patient. The diagnostic device(s) 120 and the therapeutic device(s) 110 may be selectively placed in, on, adjacent to or under the patient, and may be selectively attached to or otherwise adhered to the patient. The diagnostic device(s) 120 and/or the therapeutic device(s) 110 may communicate, e.g., with a controller, wirelessly or by a wire. The diagnostic device(s) 120 may take physiological measurements of physiological characteristics of the patient, and the therapeutic device(s) 110 may apply medical treatments to the patient. Accordingly, the medical treatment system of FIG. 1 may perform diagnostic actions such as ordering a diagnostic test or analysis, and pre-diagnostic and post-diagnostic actions including applying therapeutic treatments. The diagnostic device(s) 120 may be monitors that monitor the patient, and that analyze patient data and issue alerts. The medical treatment system of FIG. 1 is representative of a typical hospital setting, with a patient in a bed, connected to the one or more diagnostic device(s) 120 and optionally to one or more therapeutic device(s) 110. However, the medical treatment system of FIG. 1 is not limited to use in hospitals, and some or all of the elements and components of the medical treatment system of FIG. 1 may be provided to a variety of types of medical care facilities including doctor's offices, urgent care centers, nursing homes, and even in patient homes. Examples of diagnostic device(s) include a patient monitor that monitors the patient via ECG electrodes and/or an SpO2 cuff etc. Examples of therapeutic device(s) 110 include a mechanical ventilator, an intravenous therapy (IV) system, an intubation system etc. The therapeutic device(s) 110 may also issue alerts.

The medical treatment system of FIG. 1 also includes one or more access device(s) 130, radiology equipment 122, and laboratory equipment 124. The radiology equipment 122 may include medical imaging equipment including X-Ray equipment, computed tomography (CT) equipment, magnetic resonance imaging (MRI) equipment, ultrasound equipment and other forms of equipment used to perform medical imaging. The laboratory equipment 124 may include systems or devices used to diagnose a medical condition, treat a medical condition, prevent a medical condition, or rehabilitate a medical condition. The laboratory equipment 124 is separate from the diagnostic device(s) 120 and the therapeutic device(s) 110, such as by being distant from (i.e., not proximate to) the patient. The laboratory equipment 124 may be used to analyze blood, urine, saliva and other types of samples from the patient.

The medical treatment system of FIG. 1 also includes EMR system 140, a central monitoring system 150, a control system 160, an alerting system 170, a CPOE system 180, and a PACS system 190. The EMR system 140, the central monitoring system 150, the control system 160, the alerting system 170, the CPOE system 180, and the PACS system 190 may be distributed around a facility that includes the patient. For example, the EMR system 140, the central monitoring system 150, the control system 160, the alerting system 170, the CPOE system 180, and the PACS system 190 may be distributed around a hospital with the other elements and components of the medical treatment system shown in FIG. 1.

The EMR system 140 is an electronic medical record (EMR) system that is used to generate and store electronic medical records from multiple different sources so that electronic medical records from the multiple different sources are integrated and usable. The EMR system 140 may store and retrieve relevant patient data, such as all patient data used directly or indirectly in the methods described herein. The patient data from the EMR system 140 is sharable over a communications network that connects the elements and components of the medical treatment system shown in FIG. 1. All or parts of patient medical data may be stored in and available from the EMR system 140. The electronic medical record generated and stored by the EMR system 140 may be in a single format and/or a single language, may be presented in chronological order, may be electronically searchable, and may be coded in a common translatable format that should be interpretable by a clinician. A clinician as described herein is a health care worker authorized to provide care services such as diagnostic services and treatment services. Examples of clinicians include doctors and nurses.

The central monitoring system 150 may be a monitoring system that enables one or more clinician(s) to remotely monitor status of a patient from a location that is distant from (i.e., not proximate to) the patient. The central monitoring system 150 may enable clinician(s) to simultaneously remotely monitor multiple patients.

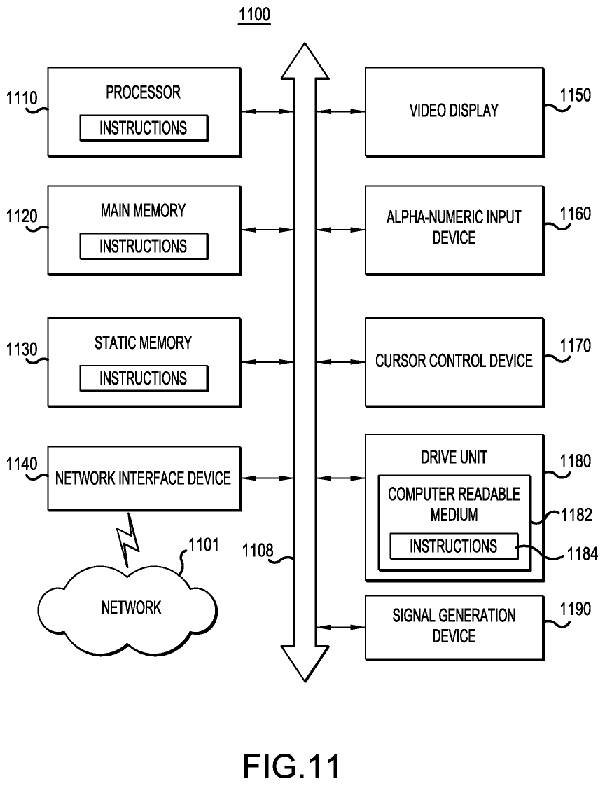

The control system 160 includes a memory that stores instructions and a processor that executes the instructions. The control system 160 may be centralized or may be distributed, and may include some or all elements and components of one or more computers or computer systems such as the computer system 1100 shown in and described with respect to FIG. 11. The control system 160 may directly or indirectly implement some or all aspects of methods and processes described herein. The control system 160 has access to the current health and treatment status of a patient from the therapeutic device(s) 110, the diagnostic device(s) 120, the access device(s) 130, the radiology equipment 122, the laboratory equipment 124, the EMR system 140, the central monitoring system 150, the alerting system 170, the CPOE system 180 and the PACS system 190 and any other relevant systems that are not depicted in FIG. 1. In other words, the control system 160 has access to patient data such as: real-time measurements, diagnostic images, labs, medical history, allergies, clinician notes, past and current pharmaceutics, past and current therapeutics, past and current procedures, and current workflow status. Real time measurements and data may include, for example, data from a mechanical ventilator or from an infusion pump. The data provided to the control system 160 may be coded, such as by using standard medical terminologies, and/or may be translatable into coded data, such as by applying natural language processing (NLP) to notes from clinicians.

The control system 160 also may store algorithms and execute algorithms by applying the algorithms to the patient data received by the control system 160. For example, algorithms may be selectively retrieved and executed based on triggers received from the alerting system 170 or directly from the diagnostic device(s) 120. The algorithms may be implemented based on formalized medical knowledge. Medical knowledge may include medical conditions and associated interventions, as well as how the associated interventions vary for different types of patients with different health characteristics. As a result, given the patient data provided to the control system 160, the algorithms may assess a patient's health status, a patient's progression, and adequacy of associated medical treatments applied to the patient as well as reasons to change the medical treatments.

The control system 160 may implement clinical decision-making. For example, given an assessment provided by applying an algorithm to patient data, the control system 160 may make a clinical decision that corresponds to a specific action such as whether or not a medical treatment should be applied to the patient in view of the patient data. The control system 160 may also determine whether additional medical data, such as additional readings from diagnostic device(s) 120, is needed to make a particular decision, and the confidence in the decision. The control system 160 may generate and store a record that can be used to explain how the decision was reached.

The control system 160 may also determine if the control system 160 can make a clinical decision such as whether a calculated confidence is sufficiently high. The control system 160 may also determine whether a clinician must make the clinical decision, and/or whether to escalate an alert when a clinician must make the clinical decision but is not available. For example, the control system 160 may escalate an alert by notifying additional clinicians such as peers, supervisors, administrators, and/or by setting off audible and/or visible alarms in a facility. If the control system 160 determines that the control system 160 can make a clinical decision, such as because a calculated confidence is sufficiently high, the control system 160 may control implementation of the clinical action. For example, the control system 160 may determine how to implement the corresponding clinical action, such as by activating or changing settings of one or more of the therapeutic device(s) 110. The control system 160 may also locate one of the therapeutic device(s) 110, and arrange to have it moved towards a patient.

The alerting system 170 may include one or more networked communications sources and distributed receivers such as mobile devices that interact over an electronic communications network. For example, the alerting system 170 may include an automated source of alerts that are sent to one or more clinicians via applications installed on mobile devices. The alerting system 170 may present alerts to the clinicians and may prompt one or more clinician(s) to respond to the alerts by entering answers and/or by travelling to patients who are the subjects of the alerts. The alerts may be sent to the alerting system 170 from the therapeutic device(s) 110 based on triggers such as to alert that the therapeutic device(s) 110 are failing or anticipating failing. The alerts may be automatically sent to the alerting system 170 from the diagnostic device(s) 120 based on triggers such as to alert that a monitored health condition of a patient is deteriorating. The alerts may also be sent to the alerting systems 170 from the mobile devices provided to clinicians, such as to alert that a clinician is unavailable.

The CPOE system 180 is a computerized physician order entry system. Clinicians can enter and receive notes and submit orders for e.g., drugs, tests or interventions via the CPOE system 180 using one of the access device(s) 130. Clinicians can use the access device(s) 130 to review patient data and receive alerts. As described herein, the medical treatment system of FIG. 1 that includes the CPOE system 180 may also review patient data and, when authorized, take the role of the clinicians and submit orders to the CPOE system 180. The alerts from the alerting system 170 may also be received via different mechanisms such as pagers.

The PACS system 190 is a picture archiving and communication system (PACS). Diagnostic images made of a patient and analyses thereof may be part of the electronic medical record and may be stored in and made available from the PACS system 190.

As an example use of the medical treatment system in FIG. 1, an automated clinical decision making (ACDM) system may analyze a patient's vital characteristics combined with the patient's current health status. When the analysis indicates that the patient urgently needs medication, the control system 160 may be alerted by the alerting system 170 and may determine that the patient should be treated with a specific dose of a specific drug. The medical treatment system in FIG. 1 may automatically retrieve and administer the specified dose of the specified compound via IV using one or more of the therapeutic device(s) 110, and the entire episode is recorded in the EMR system 140.

As set forth above, an ACDM system may improve the quality of care such as by ensuring that decisions are timely made even when a clinician is not available. An ACDM system may reduce the cost of clinical care while increasing the efficiency and quality of care. The ACDM system may supplement the care provided by clinicians, such as by making time-critical clinical decisions when a clinician is not available.

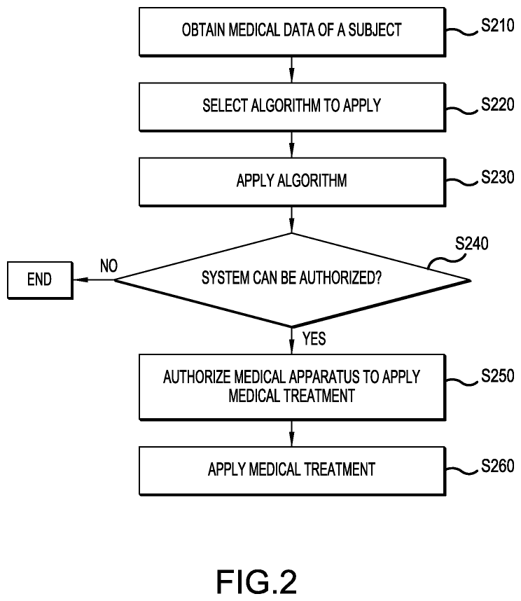

FIG. 2 illustrates a method performed by a medical treatment system, in accordance with a representative embodiment.

The method of FIG. 2 is a method for treating a patient in a medical procedure, and is performed by a system that includes a computer such as the control system 160 and a medical apparatus such as one of the therapeutic device(s) 110. The medical apparatus in the medical treatment system that performs the method of FIG. 2 is configured to apply a medical treatment to a patient when instructed to apply the medical treatment by the computer.

In FIG. 2, the method starts by obtaining medical data of a patient at S210. The medical data of the patient may be obtained by the control system 160 from the EMR system 140, such as when the EMR system 140 is the single or primary access point for accessing all patient data. The diagnostic device(s) 120 may be monitors that monitor a patient, and that periodically or continuously send real-time monitoring data as the medical data. The medical data obtained at S210 can be indicative of a medical condition to be treated. For example, the diagnostic device(s) 120 may determine that a patient's blood oxygen level is dropping below a threshold, or that a patient is suffering from the onset of a heart condition.

At S220, the method of FIG. 2 includes selecting one or more algorithms to apply. The algorithms may be selected by the control system 160 from a multitude of algorithms stored in the memory 161, and the selection may be based on the type of the medical data and interpretation of the medical data obtained at S210 together with additional medical data of the patient obtained from the EMR system 140. In the medical treatment system of FIG. 1, multiple algorithms may be running continuously and in parallel for the same patient and for multiple different patients, and each algorithm that is run may be elected in accordance with a process that includes functionality such as S220.

At S230, the method of FIG. 2 includes applying the algorithm. In some embodiments, S230 may include selecting multiple algorithms. The selected algorithms may be applied to the medical data to identify a medical treatment to remedy the medical condition indicated by the medical data obtained at S210. A “remedy” as described herein may include an action to attempt to alleviate or otherwise improve the medical condition. For example, the selected algorithm may identify a new medication, a changed dosage of a medication, an increase or decrease of oxygen supply, or another form of medical treatment to be applied. The algorithm applied at S230 may also determine other actions needed, such as ordering a medication for the patient.

At S240, the method of FIG. 2 includes determining whether the system that performs the method of FIG. 2 can be authorized to perform an action identified at S230 based on the medical data obtained at S210. The determining at S240 may involve a lookup table of authorized clinical actions. The authorization that is the subject of S240 may be determined after obtaining the medical data of the patient at S210 without further instructions from a clinician.

At S250, the method of FIG. 2 includes instructing the medical apparatus to apply the medical treatment when the system is authorized to apply the medical treatment based on the determination at S240. The instruction at S250 may include providing a command to take a specific action. For example, at S250 the control system 160 may instruct one of the therapeutic devices(s) 110 to initiate a treatment or change a level of a medical treatment to apply the medical treatment to the patient. Instructing at S250 may be performed by sending a command to one or more of the therapeutic device(s) 110.

At S260, the medical apparatus applies the medical treatment based on instructions from the control system 160. For example, based on the instructions at S250, one of the therapeutic device(s) 110 may apply the medical treatment to the patient at S260. The medical treatment is applied to the patient by the medical apparatus at S260 based on the control system 160 instructing the medical apparatus to apply the medical treatment to the patient.

In the method of FIG. 2, the medical data may be real-time data, and the medical data may raise time-critical concerns that must be addressed. Real-time data may include, for example, data from a mechanical ventilator and data from an infusion pump. As a result, the control system 160 may select and apply an algorithm to determine whether the medical treatment system of FIG. 1 can be used to automatically identify and apply a medical treatment, or whether an attempt to locate and contact an available clinician must first be made. Sub-processes that occur between S240 and S250 may identify clinicians who are authorized to make the clinical decision for the medical treatment, and the conditions under which the control system 160 is authorized to make the clinical decision. Before allowing the control system 160 to authorize the medical treatment, the control system 160 may track the location and availability of each identified clinician. The identified clinicians may include telemedicine staff. The tracking by the control system 160 may also include locations of each patient in a facility, and various of the medical apparatuses that may be used as therapeutic device(s) 110.

Moreover, sub-processes between S240 and S250 may include assessing if an authorized clinician is available to make a time-critical clinical decision. If the authorized clinician is available, the sub-processes may include attempting to contact the authorized clinician and establishing an established period of time for response. The absence of a response to an alert sent to an authorized clinician within the required period of time for response may trigger the control system 160 to make the clinical decision and authorize the medical treatment. Similarly, if a sub-process indicates that no authorized clinician is available to make the decision and the control system 160 may be authorized to make the clinical decision, the control system 160 may proceed with making the clinical decision and instructing the therapeutic device(s) 110 to apply the optimal medical treatment. Additionally, the sub-processes may allow for an override by a clinician to countermand an instruction by the control system 160.

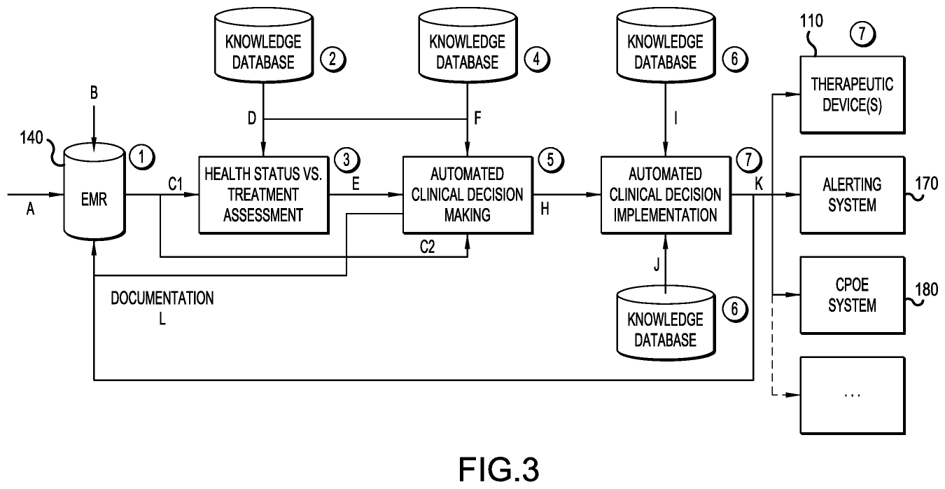

FIG. 3 illustrates a hybrid of a medical treatment system and a method performed by the medical treatment system, in accordance with a representative embodiment.

In FIG. 3, seven elements are labelled from “1” to “7”. The first element is the EMR system 140 labelled as “1” (hereinafter “element 1”). The second element is a knowledge database labelled as “2” (hereinafter “element 2”). The third element is algorithms labelled as “3” (hereinafter “element 3”). The fourth element is another knowledge database labelled as “4” (hereinafter “element 4”. The fifth element is an automated clinical decision-making mechanism labelled as “5” (hereinafter “element 5”). The sixth element is another knowledge database labelled as “6” (hereinafter “element 6”). The seventh element is an implementation of an automated clinical decision using the therapeutic device(s) 110 labelled as “7” (hereinafter “element 7”). As will be evident, element 2, element 4 and element 6″ may be implemented using the memory 161 of the control system 160. Element 3 may be stored in the memory 161 of the control system 160, and may be executed by the processor 162 of the control system 160. Element 5 may be implemented by the processor 162 of the control system. Element 7 may be implemented by the processor 162 of the control system 160 and by the therapeutic device(s) 110.

The input to the medical treatment system in FIG. 3 is new (initial) patient data and “live’ patient data such as treatment data. The output from the medical treatment system in FIG. 3 is decisions with documentation of associated actions that is automatically generated and which is stored in, e.g., the EMR system 140. The output from the medical treatment system in FIG. 3 can encompass data used to make a clinical decision, the decision that is made, an explanation of how the decision is made, action(s) taken, and so on.

The medical treatment system in FIG. 3 includes element 1. The EMR system 140 includes one or more mechanisms to access the current health and treatment status of a patient. The electronic medical records in the EMR system 140 may include real-time measurements, diagnostic images, labs, medical history, allergies, clinician notes, current pharmaceutics, current therapeutics, current procedures, and current workflow status such as whether a patient is in transit. The electronic medical records in the EMR system 140 may be coded using standard medical terminologies, or may be translatable into coded data such as by clinician notes that are translatable via natural language processing (NLP)). As shown in FIG. 3, the EMR system 140 receives new patient data including new treatment data at step A and new (initial) patient data at step B. At step C1, the EMR system 140 provides the new patient data to element 3. At step C2, the EMR system 140 provides the new patient data to element 5. The EMR system 140 may receive data from any of the elements and components of the medical treatment system in FIG. 1.

The medical treatment system in FIG. 3 also includes element 2. The knowledge database of element 2 includes one or more mechanisms to enable algorithmic assessment of a patient's health status and progression and of the adequacy of associated treatments versus needs to changes the associated treatments. The knowledge database of element 2 stores knowledge that specifies how data from the EMR system 140 can and/or should be interpreted. Information sources that can be used for providing data to the EMR system 140 include medical, biomedical, genetic, and clinical, ontologies, terminologies and vocabularies such as Gene Ontology (GO), Logical Observational Identifiers, Names, and Codes (LOINC), International Classification of Diseases (ICD) 9 or 10, Systemized Nomenclature of Medicine-Clinical Terms (SNOMED-CT), etc.), deterministic, probabilistic, neural, and/or physiological models, and clinical rules. Other information sources that can be used to implement the EMR system 140 include organ status indicators, health status predictors, and medical imaging analytics. At step D, the knowledge database of element 2 provides medical knowledge to the algorithms of element 3.

The medical treatment system of FIG. 3 also includes algorithms of element 3. The one or more algorithms of element 3 are used to implement the treatment assessments based on medical data indicative of health status of the patient as described herein. The assessments implemented using the algorithms may include a response to any treatment actions. At step E, the algorithms of element 3 provide recommended treatment updates to the automated clinical decision-making mechanism of element 5. The algorithms of element 3 may be implemented by one or more programs that combine the knowledge from the EMR system 140 with patient data to obtain patient-specific health status and treatment indicators. Programs used to implement the knowledge database of element 2 may include inference engines of various types and programmed algorithms to combine the results of intermediate inferences. The knowledge database of element 2 may also store and provide a response to treatment actions.

The medical treatment system of FIG. 3 includes a knowledge database of element 4. The knowledge database of element 4 may include facilities to indicate what patient data is mandatory and what patient data is optional. Optional data may be data that, if obtained, is expected to increase confidence. Algorithms for reaching clinical decisions based on decision-making knowledge may, for example, be codified as a set of rules combined with a Bayesian network. The algorithms may be obtained from a combination of machine learning and expert knowledge. Confidence may be explicitly encoded in the rules of algorithms. Furthermore, an explanation of how a decision was reached may be obtained by tracing which rules fired and the data that caused the rules to fire. Algorithms of element 3 may also include algorithms for extracting explanations from Bayesian networks.

The medical treatment system of FIG. 3 also includes an automated clinical decision-making mechanism of element 5. This element uses one or more mechanisms to formalize clinical decision making knowledge in such a way that given the above assessment, a computer can make a clinical decision that corresponds to a specific action. This formalization includes one or more mechanisms to specify what patient data is required to make a particular decision, the confidence in the decision, and an explanation of how the decision was reached. At step F, the knowledge database of element 4 provides clinical decision-making knowledge to the automated clinical decision-making mechanism of element 5.

The automated clinical decision-making mechanism of element 5 may include the control system 160 of FIG. 1. The automated clinical decision-making mechanism may check if a clinical decision can be made by the control system 160, such as if all required data and knowledge is available to make a decision and if the confidence in the clinical decision is sufficiently high. If the automated clinical decision-making mechanism in FIG. 3 determines that the clinical decision can be made, the automated clinical decision-making mechanism may proceed with making the clinical decision. At step H, the automated clinical decision-making mechanism of element 5 provides the clinical decision for implementation by element 7. The automated clinical decision-making mechanism of element 5 may include one or more programs that execute the algorithms of element 3. The programs of the knowledge database of element 4 may include a rules engine and a Bayesian network (inference) engine.

The medical treatment system of FIG. 3 further includes knowledge databases of element 6. The knowledge databases of element 6 enable the medical treatment system to know if corresponding actions can automatically be taken, and if so, how. The knowledge databases of element 6 may store knowledge of locations of the therapeutic device(s) 110 and availability of the therapeutic device(s) 110. Element 6 may provide a parameterized mapping of possible decisions to possible actions. Each action may be specified as a specific, parameterized interaction with the components and elements of the medical treatment system of FIG. 1, and may range from changing the setting of a ventilator to administering a drug or to calling a code. Examples of parameters include patient ID, patient location, device ID, device location, drug ID, drug dose.

At step I the knowledge databases of element 6 provide the clinical actions knowledge for implementation by element 7. At step J the knowledge databases of element 6 provide indications of which of the therapeutic device(s) 110 are available for action implementation by element 7.

The medical treatment system of FIG. 3 includes implementation of element 7. Element 7 provides for taking the corresponding action determined by the control system 160, if possible. At step K, the action to be taken by element 7 is specified. The action is instructed or reported to the therapeutic device(s) 110 or to the alerting system 170 or to the CPOE system 180.

At step L, documentation of actions taken by the medical treatment system of FIG. 3 are reported to the EMR system 140. Documentation is taken from element 7.

Examples of actions that may be taken by the medical treatment system in FIG. 3 include applying or modifying therapeutics through a qualified and available medical device. For example, an action that may be authorized may include changing ventilator settings and/or starting, stopping or changing dosage of an intravenous drug. Another example of the actions that a medical treatment system in FIG. 3 may make include placing an order for delivery of pharmaceuticals to the bedside; moving a urine sample to the laboratory equipment 124, diagnostic imaging by the radiology equipment 122 and so on. Yet another example of the actions that a medical treatment system in FIG. 3 may make include calling a code, such as a code blue for a cardiopulmonary arrest.

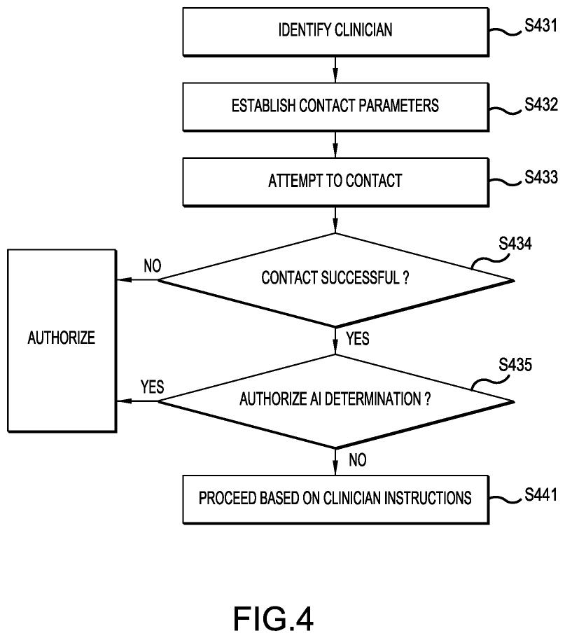

FIG. 4 illustrates another method performed by a medical treatment system, in accordance with a representative embodiment.

In FIG. 4, the process starts at S431 by identifying a clinician for making a clinical decision and/or taking a clinical action. The clinician may be identified based on the patient data including identity of the patient, medical condition of the patient, availability of the clinician, role of the clinician, and other relevant information. Additionally, S431 may include identifying multiple clinicians.

At S432, the method of FIG. 4 includes establishing contact parameters. Contact parameters may include how and whether to contact the clinician, how much time the clinician may be given to respond, and options that may be presented to the clinician if the clinician is contacted and is able to respond.

At S433, the method of FIG. 4 includes attempting to contact the clinician. The attempt may be made by a messaging service, a phone call, a page, or any other appropriate form of modern electronic communications.

At S434, a determination is made whether the contact is successful. If contact is not successful (S434=No), the control system 160 may authorize the ACDM system to make the clinical decision and/or take the clinical action.

If the attempt to contact the clinician is successful (434=Yes), another determination is made at S435 whether the clinician has authorized an artificial intelligence (AI) determination by the control system 160. The authorization from a clinician may be from an individual clinician, a team of clinicians, and possibly from non-clinical staff. If the AI determination is authorized (S435=Yes), the control system 160 may authorize the clinical action such as by initiating the clinical action. If the AI determination is not authorized (S435=No), the control system 160 may proceed based on the clinician's instructions at S441 or may simply ‘stand down’.

As described above, in FIG. 4 time-sensitive clinical decisions may be selectively left in the hands of clinicians. If the clinicians cannot be contacted, or if the AI determination has been authorized, the control system 160 may be responsible for making the clinical decision and taking the associated clinical action.

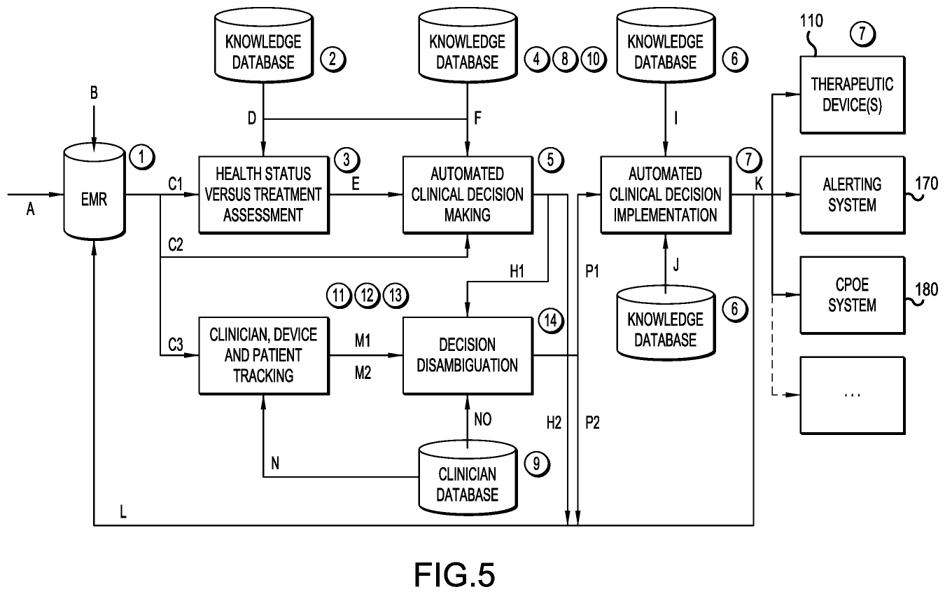

FIG. 5 illustrates another hybrid of a medical treatment system and a method performed by the medical treatment system, in accordance with a representative embodiment, in accordance with a representative embodiment.

The medical treatment system in FIG. 5 is a refinement of the medical treatment system of FIG. 3 In FIG. 5, additional elements labelled “8” through “14” include a knowledge database labelled as “8” (hereinafter “element 8”), a clinician database labelled as “9” (hereinafter “element 9”), a knowledge database labelled as “10” (hereinafter “element 10”), a clinician tracking mechanism labelled as “11” (hereinafter “element 11”), a clinician disambiguation mechanism labelled as “12” (hereinafter “element 12”), an override mechanism labelled as “13” (hereinafter “element 13”), and a knowledge acquisition and management mechanism labelled as “14” (hereinafter “element 14”). FIG. 5 illustrates how the additional elements labelled “8” through “14” can be added to disambiguate clinical decisions and the associated actions. Disambiguation is a term used herein to describe clarifying which of the control system 160 or a clinician will be responsible for making a clinical decision and taking action.

The knowledge database of element 8 may provide an indication of the relative urgency of making a clinical decision, such as whether a decision is time critical and how much time can be afforded for a time-critical clinical decision. The knowledge database of element 8 may be implemented by systematically encoding logic that determines the relative urgency of actions to be taken.

The clinician database of element 9 may be implemented by using mapping in a lookup table. The mapping may map clinicians to authorized clinical actions which the clinicians are authorized to make. The mapping may be implemented, for example, by mapping clinician roles such as triage nurse or respiratory therapist, to actions or to classes of actions. The mapping may specify clinician roles (e.g., triage nurse, respiratory therapist) to actions or classes of actions.

The knowledge database of element 10 provides a mapping of ACDM decisions to authorized clinical actions. The mapping in the knowledge database of element 10 may formalize and specify which clinical decisions the ADCM system is authorized to make. Element 10 may be implemented using mapping in a lookup table of clinical decisions which may be made by the control system 160 under predetermined conditions.

The clinician tracking mechanism of element 11 provides a mechanism for tracking location and availability of clinicians. For example, coding and tracking staff location may involve a location tracking system that tracks staff locations using an associated map of the medical facility that includes the medical treatment system of FIG. 5. The location tracking may use radio frequency identification (RFID) or cameras. Staff availability may also be inferred from image analysis from images provided by fixed cameras, accelerometers or other mechanisms that reflect physical activity, body-worn cameras, microphones, speech to text translators, natural language processing (NLP) to analyze language, and other mechanisms to infer current activity. Staff availability may be inferred from proximity to patient, current activity, and the assessed needs of other patients. Availability may also be assumed, such as for telemedicine staff who are on duty.

The device tracking mechanism of element 12 may be implemented to track the location and availability of each of the therapeutic device(s) 110 and to automatically move available therapeutic device(s) to patients. The knowledge database of element 10 may be implemented using knowledge of which of the therapeutic device(s) 110 are available to treat a patient. One of the therapeutic device(s) 110 connected to a patient may be associated with that patient via a user interface. The location of all therapeutic device(s) 110 may be tracked via RFID and/or camera. The availability may be checked by one of the therapeutic device(s) 110 i) not being in motion and ii) not being associated with a patient. Optionally, one of the therapeutic device(s) 110 may be automatically moved to a patient via a robotic locomotion and navigation system. An ACDM system may track devices using element 13 in order to know which devices are available and appropriate for treating a patient.

The patient tracking mechanism of element 13 may be implemented to formalize and track the location of each patient. Techniques for tracking the location of clinicians in element 11 may also be used for tracking the location of patients in element 13. For example, tracking the location of beds may be used since patients are often in or around a bed. Patient-to-bed mapping may be used in element 13 when beds are tracked.

Element 14 assesses if an onsite or remote authorized clinician is available for making a time-critical clinical decision that the system detected as having to be made. Availability or non-availability may be inferred from a response or absence of a response to an alert sent to authorized clinicians within a certain amount of time, or may be communicated by the clinician(s) in response to the alert. A negative response or absence of a response may be used to disambiguate that the control system 160 is responsible for making a clinical decision, such as when no authorized clinician is available to make a time-critical clinical decision while the control system 160 is authorized and capable of making the clinical decision.

Element 14 may be implemented by constructing a logical hierarchy of characteristics of clinicians, such as expertise, specialty, experience. The logical hierarchy may also be constructed for the control system 160 for comparative purposes, such as to clarify how confident the control system 160 may be in making a clinical decision based on the medical data available to the control system 160. The clinician tracking mechanism of element 11 may be used by the disambiguation mechanism of element 14 to assess if an onsite or remote authorized clinician is available to make a time-critical clinical decision that the control system 160 detects as having to be made.

The clinician database of element 9 may be used to identify which clinicians are authorized to make a decision, the clinician tracking mechanism of element 11 may locate the clinicians authorized to make the clinical decision, and the disambiguation mechanism of element 14 may assess the location and availability of these clinicians, and prioritize or otherwise rank the available clinicians (if any) such as by proximity to the patient. Notifications may be sent to the most suitable clinician(s), and the control system 160 may wait for an established period of time for a response. In the case of a negative response or absence of a response, the disambiguation mechanism of element 14 may indicate that the control system 160 may proceed with making and implementing the clinical decision.

Other elements that may be present in embodiments based on FIG. 5 may include a secondary disambiguation mechanism that disambiguates how a decision is to be made when a clinician is authorized to make the decision and the ACDM system is also authorized to make the decision. For example, the ACDM system may be authorized in advance to make a predetermined type of decision even when a clinician is also authorized and available to make the decision. The secondary disambiguation mechanism may formalize both which clinicians are authorized to make a given decision and how suitable the clinicians are to make the decision. For example, a secondary disambiguation mechanism may consider expertise, specialty, experience (e.g., years in the field and years in their current role) A suitability determination may also be made for the ACDM system for some types of decisions. When the decision is to be made and both the ACDM system and an appropriate clinician are available, suitability may be used to determine whether the ACDM system or the clinicians makes the decision.

Another element that may be used based on the embodiment of FIG. 5 is a mechanism to provide for clinicians to override clinical decisions made or about to be implemented by the control system 160. The override mechanism may be implemented using a user interface on a computer or medical device networked to the control system 160, or using an emergency ‘stop’ button proximate to the patient such as next to a patient bed. The override mechanism stops the control system 160 from implementing a particular action and is documented in the EMR system 140, along with what action was overridden and by which clinician. The clinician may be identified in the case of the emergency ‘stop” button by a camera activated by the ‘stop” button and image recognition software. The control system 160 may also confirm whether the clinician pushing the ‘stop’ button has authority to override the control system 160, to prevent use by unauthorized persons such as visitors.

An additional element that may be provided to embodiments based on FIG. 5 may be implemented to learn, enter, review, edit, manage and deploy the algorithms and medical data described herein. The control system 160 may rely on large amounts of codified knowledge, including medical knowledge and knowledge about staffing, medical apparatuses, hospital layout. Since a part of the knowledge consumed by the control system 160 may be facility specific and part of the knowledge consumed by the control system 160 may be subject to change, the additional element may provide supervisory functions to update existing algorithms and knowledge, including whether a clinician or other human is required to approve any particular update.

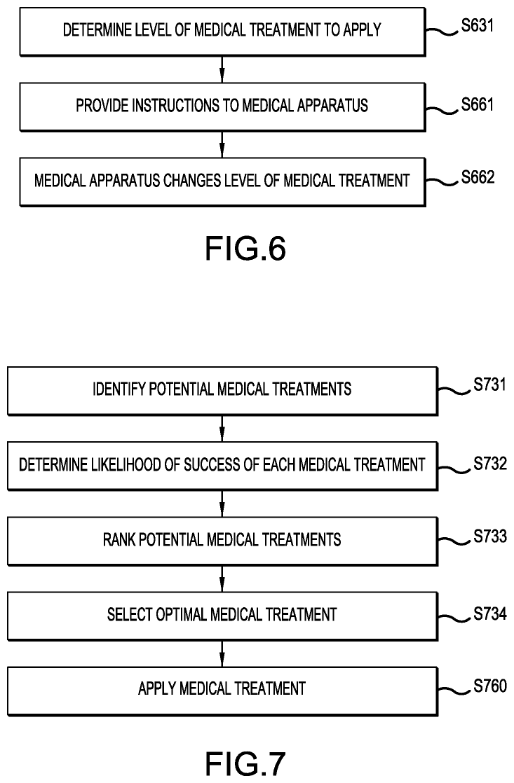

FIG. 6 illustrates another method performed by a medical treatment system, in accordance with a representative embodiment.

The method of FIG. 6 starts with determining a level of medical treatment to apply at 5631. For example, the level may be a level of oxygen, an absolute or relative (i.e., concentration) of a compound, or another type of level that may vary based on a change in patient health condition.

At S661, the method of FIG. 6 includes providing instructions to the medical apparatus based on the determined level of medical treatment from S631. For example, instructions may be to start providing oxygen to a patient, or to increase the rate at which oxygen is provided.

At S662, the medical apparatus changes the level of medical treatment. The medical apparatus may be one of the therapeutic device(s) 110, and the instructions may be provided from the control system 160.

FIG. 7 illustrates another method performed by a medical treatment system, in accordance with a representative embodiment.

The method of FIG. 7 starts at S731 by identifying potential medical treatments. The identification at S731 may be triggered by receipt of new medical data at the control system 160.

At S732, the method of FIG. 7 includes determining likelihood of success of each medical treatment identified at S731. The likelihood of success may be determined by an algorithm that weighs available medical data for the patient in order to determine an optimal course of action such as an optimal medical treatment. At S733, the potential medical treatments are ranked relative to one another, and at S734 the optimal medical treatment may be selected by the algorithm run by the control system 160. That is, at S734, the method of FIG. 7 may include identifying the medical treatment as an optimal medical treatment based on rankings of multiple potential medical treatments generated from applying an algorithm to received medical data.

At S760, the optimal medical treatment is applied by one of the therapeutic devices(s) 110. The optimal medical treatment may be applied at S760 based on instructions from the control system 160.

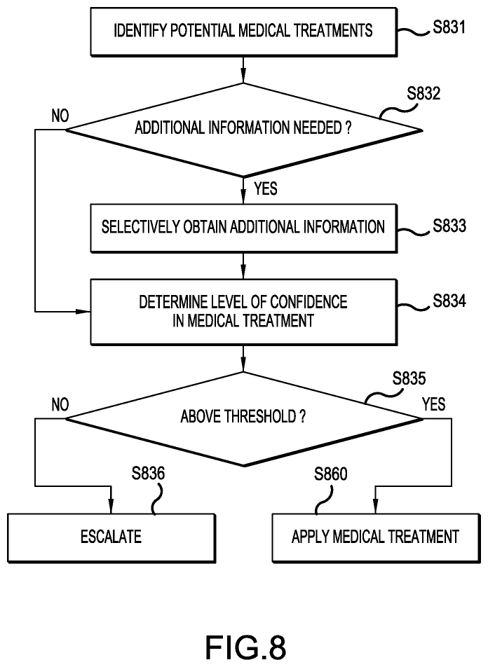

FIG. 8 illustrates another method performed by a medical treatment system, in accordance with a representative embodiment.

The method of FIG. 8 begins at S831 by identifying potential medical treatments. The potential medical treatments may be identified at S831 based on receipt of new medical data at the control system 160.

At S831, the control system 160 determines whether additional information is needed in order to identify a proper medical treatment to apply. The determining at S831 result in identifying additional information required in order to determine whether the ACDM system can be authorized to apply the medical treatment to the patient. If no additional information is needed (S832=No), at S834 the control system 160 may determine levels of confidences in the medical treatment(s) identified at S831. If additional information is needed (S832=Yes), the control system 160 may request the additional information from any of the information sources described herein at S833. For example, the control system may request additional information from a blood pressure or heartrate monitor as one of the diagnostic device(s) 120 to determine a specific current condition of the patient.

At S834, the level of confidence a potential medical treatment is identified. For example, if only one potential medical treatment is identified at S831 for a particular medical condition, the level of confidence in the potential medical treatment may be relatively high. On the other hand, if additional relevant information is needed but not available, such as from the radiology equipment 122 or the laboratory equipment 124, the level of confidence in the potential medical treatment may be relatively low.

At S835, the control system 160 determined whether the determined level of confidence is above a threshold. If the determined level of confidence is above a threshold (S835=Yes), the control system 160 may send instructions to one of the therapeutic device(s) 110 to apply the medical treatment at S860. However, if the level of confidence is not above the threshold (S835=Now), the control system 160 may escalate the medical concern in the medical treatment system at S836. The escalation at S836 may be due to the relative urgency of the medical concern, the relative unavailability of a clinician to make a clinical decision, and the relative lack of confidence of the control system 160 in its own clinical decision.

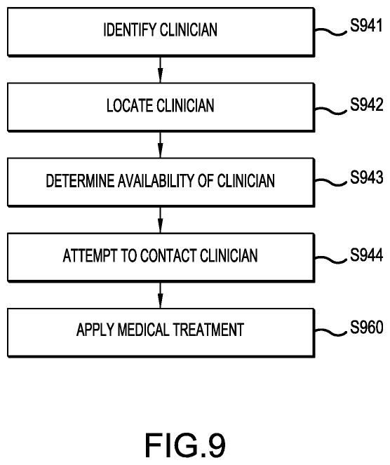

FIG. 9 illustrates another method performed by a medical treatment system, in accordance with a representative embodiment.

The process of FIG. 9 begins at S941 by identifying a clinician. The clinician may be identified by an algorithm run by the control system 160 based on receipt of particular medical data. At S942, the method of FIG. 9 includes locating the clinician identified at S941. The clinician may be located based on an RFID tag carried by the clinician in the facility that includes the medical treatment system of FIG. 1.

At S943, the availability of the clinician is identified. For example, the control system 160 may determine whether the clinician is at work and, if so, attending to another patient or at a relatively great distance from the patient requiring attention now. At S944, the control system 160 attempts to contact the clinician, such as by generating and sending a message to the clinician, paging the clinician in a portion of the facility in which the clinician is located, or calling the clinician.

At S960, the medical treatment is applied. The medical treatment is applied at S960 by the clinician if the clinician is available, or based on instructions from the control system 160 if the clinician is unavailable and the control system 160 is relatively confident in a proposed medical treatment and the ACDM system is capable of applying the treatment.

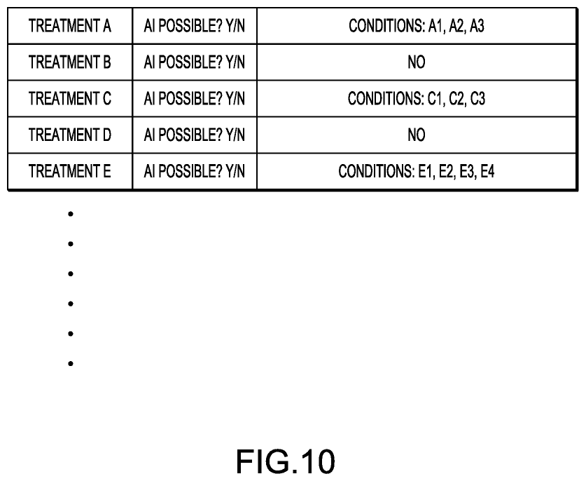

FIG. 10 illustrates a data set used as an input for a medical treatment system, in accordance with a representative embodiment.

The data set of FIG. 10 shows whether artificial implementations are possible for particular treatments that may be identified by the control system 160. For example, treatment B may correspond to a broken leg that must be reset by hand by a skilled physician or therapist, and which cannot be appropriately treated solely by instructions from the control system 160. On the other hand, treatment A may correspond to a medical issue that can be remedied automatically based on instructions from the control system 160. As a result, treatment A may correspond to a particular algorithm run by the control system 160 based on specific conditions A1, A2, A3. The control system 160 may apply the predetermined algorithm to the medical data including the conditions A1, A2, A3 in order to generate optimized conditions for the treatment A. If the control system 160 is confident that treatment A will properly remedy the medical condition at issue, the control system 160 may send instructions to one of the therapeutic device(s) 110 to apply the medical treatment to the patient.

FIG. 11 illustrates a computer system in a medical treatment system, in accordance with another representative embodiment.

The computer system 1100 of FIG. 11 shows a complete set of components for a communications device or a computer device. However, a “controller” as described herein may be implemented with less than the set of components of FIG. 11, such as by a memory and processor combination. The computer system 1100 may include some or all elements of one or more component apparatuses in a medical treatment system herein, although any such apparatus may not necessarily include one or more of the elements described for the computer system 1100 and may include other elements not described.

Referring to FIG. 11, the computer system 1100 includes a set of software instructions that can be executed to cause the computer system 1100 to perform any of the methods or computer-based functions disclosed herein. The computer system 1100 may operate as a standalone device or may be connected, for example, using a network 1101, to other computer systems or peripheral devices. In embodiments, a computer system 1100 performs logical processing based on digital signals received via an analog-to-digital converter.

In a networked deployment, the computer system 1100 operates in the capacity of a server or as a client user computer in a server-client user network environment, or as a peer computer system in a peer-to-peer (or distributed) network environment. The computer system 1100 can also be implemented as or incorporated into various devices, such as the control system 160 in FIG. 1, a stationary computer, a mobile computer, a personal computer (PC), a laptop computer, a tablet computer, or any other machine capable of executing a set of software instructions (sequential or otherwise) that specify actions to be taken by that machine. The computer system 1100 can be incorporated as or in a device that in turn is in an integrated system that includes additional devices. In an embodiment, the computer system 1100 can be implemented using electronic devices that provide voice, video or data communication. Further, while the computer system 1100 is illustrated in the singular, the term “system” shall also be taken to include any collection of systems or sub-systems that individually or jointly execute a set, or multiple sets, of software instructions to perform one or more computer functions. As illustrated in FIG. 11, the computer system 1100 includes a processor 1110. The processor 1110 may be considered a representative example of the processor 1112 of the control system 160 in FIG. 1 and executes instructions to implement some or all aspects of methods and processes described herein. The processor 1110 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1110 is an article of manufacture and/or a machine component. The processor 1110 is configured to execute software instructions to perform functions as described in the various embodiments herein. The processor 1110 may be a general-purpose processor or may be part of an application specific integrated circuit (ASIC). The processor 1110 may also be a microprocessor, a microcomputer, a processor chip, a controller, a microcontroller, a digital signal processor (DSP), a state machine, or a programmable logic device. The processor 1110 may also be a logical circuit, including a programmable gate array (PGA), such as a field programmable gate array (FPGA), or another type of circuit that includes discrete gate and/or transistor logic. The processor 1110 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1100 further includes a main memory 1120 and a static memory 1130, where memories in the computer system 1100 communicate with each other and the processor 1110 via a bus 1108. Either or both of the main memory 1120 and the static memory 1130 may be considered representative examples of the memory 191 of the control system 160 in FIG. 1, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1120 and the static memory 1130 are articles of manufacture and/or machine components. The main memory 1120 and the static memory 1130 are computer-readable mediums from which data and executable software instructions can be read by a computer (e.g., the processor 1110). Each of the main memory 1120 and the static memory 1130 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RANI memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1100 further includes a video display unit 1150, such as a liquid crystal display (LCD), an organic light emitting diode (OLED), a flat panel display, a solid-state display, or a cathode ray tube (CRT), for example. Additionally, the computer system 1100 includes an input device 1160, such as a keyboard/virtual keyboard or touch-sensitive input screen or speech input with speech recognition, and a cursor control device 1170, such as a mouse or touch-sensitive input screen or pad. The computer system 1100 also optionally includes a disk drive unit 1180, a signal generation device 1190, such as a speaker or remote control, and/or a network interface device 1140.

In an embodiment, as depicted in FIG. 11, the disk drive unit 1180 includes a computer-readable medium 1182 in which one or more sets of software instructions 1184 (software) are embedded. The sets of software instructions 1184 are read from the computer-readable medium 1182 to be executed by the processor 1110. Further, the software instructions 1184, when executed by the processor 1110, perform one or more steps of the methods and processes as described herein. In an embodiment, the software instructions 1184 reside all or in part within the main memory 1120, the static memory 1130 and/or the processor 1110 during execution by the computer system 1100. Further, the computer-readable medium 1182 may include software instructions 1184 or receive and execute software instructions 1184 responsive to a propagated signal, so that a device connected to a network 1101 communicates voice, video or data over the network 1101. The software instructions 1184 may be transmitted or received over the network 1101 via the network interface device 1140.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing

Accordingly, a medical treatment system enables automated determinations for an optimized type of an interventional procedure such as a biopsy of a lung. Nevertheless, a medical treatment system is not limited as an application to lungs, and instead is applicable to other organs for which multiple biopsy approaches may be feasible. Similarly, a medical treatment system is not limited to biopsies, and instead is applicable to other types of interventional procedures such as ablation or other types of therapeutic interventions in which multiple approaches may be feasible. Similarly, a medical treatment system is not limited to treatment per se but can also automate auxiliary tasks such as ordering medication or a diagnostic test for a patient.

Although a medical treatment system has been described with reference to several exemplary embodiments, it is understood that the words that have been used are words of description and illustration, rather than words of limitation. Changes may be made within the purview of the appended claims, as presently stated and as amended, without departing from the scope and spirit of a medical treatment system in its aspects. Although a medical treatment system has been described with reference to particular means, materials and embodiments, a medical treatment system is not intended to be limited to the particulars disclosed; rather a medical treatment system extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system for treating a patient in a medical procedure, comprising:
a computer comprising a memory that stores instructions and a processor that executes the instructions; and
a medical apparatus configured to apply a medical treatment to the patient when instructed to apply the medical treatment by the computer, wherein,
when executed by the processor, the instructions cause the system to:
obtain medical data of the patient indicative of a medical condition to be treated;
select an algorithm and apply the algorithm to the medical data to identify the medical treatment to remedy the medical condition;
determine whether the system is authorized to apply the medical treatment to the patient; and
when the system is authorized to apply the medical treatment to the patient, instruct the medical apparatus to apply the medical treatment to the patient,
wherein the medical apparatus applies the medical treatment to the patient based on the computer instructing the medical apparatus to apply the medical treatment to the patient.

2. The system of claim 1, wherein the medical data includes real-time data updated periodically in real-time from a monitor that monitors the patient.

3. The system of claim 1, wherein the instructions cause the system further to:
identify and attempt to contact over a communications network a clinician authorized to apply the medical treatment to remedy the medical condition;
establish an established period of time in which the clinician must provide instructions; and
determine that the system is authorized to apply the medical treatment to the patient without instructions from the clinician when the clinician does not provide instructions within the established period of time.

4. The system of claim 1,
wherein the medical apparatus treats the patient by changing a level of the medical treatment already being supplied to the patient based on an instruction from the computer.

5. A method for treating a patient in a medical procedure, comprising:
obtaining, via a computer system comprising a memory that stores instructions and a processor that executes the instructions, medical data of the patient indicative of a medical condition to be treated;
selecting, by the computer system, an algorithm and applying the algorithm to the medical data to identify a medical treatment to remedy the medical condition;
determining, by the computer system, whether a system that includes a medical apparatus and the computer system can be authorized to apply the medical treatment to the patient; and
when the system can be authorized to apply the medical treatment to the patient, instructing the medical apparatus to apply the medical treatment to the patient,
wherein the medical apparatus applies the medical treatment to the patient based on the computer system instructing the medical apparatus to apply the medical treatment to the patient.

6. The method of claim 5, further comprising:
identifying the medical treatment as an optimal medical treatment based on rankings of a plurality of medical treatments generated from applying the algorithm to the medical data.

7. The method of claim 5, wherein a determination whether the medical apparatus can be instructed to apply the medical treatment to the patient is made without instructions from a clinician.

8. The method of claim 5, further comprising:
determining, by the algorithm, a likelihood of success of the medical treatment.

9. The method of claim 5, further comprising:
determining, by the algorithm, additional medical data required in order to determine whether the medical apparatus can be instructed to apply the medical treatment to the patient; and
selectively obtaining the additional medical data,
wherein the determining whether the system can be authorized to apply the medical treatment to the patient is performed at least partially based on the additional medical data.

10. The method of claim 5, further comprising:
determining a level of confidence in the medical treatment,
wherein the determining whether the system can be authorized to apply the medical treatment to the patient is performed based on the level of confidence in the medical treatment.

11. The method of claim 5, further comprising:
identifying a clinician authorized to apply the medical treatment to the patient; and
attempting to contact the clinician over a communications network,
wherein the instructing the medical apparatus to apply the medical treatment to the patient is based on being unable to contact the clinician over the communications network.

12. The method of claim 5, further comprising:
locating, based on obtaining the medical data, a clinician authorized to apply the medical treatment to the patient; and
determining availability of the clinician.

13. The method of claim 5, further comprising:
instructing the medical apparatus to apply the medical treatment to the patient based on instructions from a clinician authorized to apply the medical treatment to the patient.

14. A tangible non-transitory computer readable storage medium that stores a computer program, the computer program, when executed by a processor of a computer, causing a system that includes the tangible non-transitory computer readable storage medium to:
obtain medical data of a patient indicative of a medical condition to be treated;
select an algorithm and apply the algorithm to the medical data to identify a medical treatment to remedy the medical condition;
determine whether a medical apparatus can be instructed to apply the medical treatment to the patient; and
when the medical apparatus can be instructed to apply the medical treatment to the patient, instruct the medical apparatus to apply the medical treatment to the patient,
wherein the medical apparatus applies the medical treatment to the patient based on the computer instructing the medical apparatus to apply the medical treatment to the patient.

15. The tangible non-transitory computer readable storage medium of claim 14,
wherein the computer program further causes the system to:
identify a plurality of clinicians authorized to apply the medical treatment to the patient;
attempt to locate each of the plurality of clinicians authorized to apply the medical treatment to the patient;
determine, based on locating at least one of the plurality of clinicians, proximity of the at least one of the plurality of clinicians to the medical apparatus;
attempt to contact the at least one of the plurality of clinicians over a communications network based the proximity of the at least one of the plurality of clinicians to the medical apparatus, and determine whether the medical apparatus can be instructed to apply the medical treatment to the patient only when the at least one of the plurality of clinicians cannot be contacted over a communications network.
